(19) United States
(12) Patent Application Publication
(10) Pub. No.: US 2008/0021454 A1
(43) Pub. Date: Jan. 24, 2008

(54) SACRAL OR ILIAC CONNECTOR

(75) Inventors: Nam T. Chao, Marlborough, MA (US); Peter Newton, San Diego, CA (US); Randal Betz, Ocean City, NJ (US); Tim Mondeau, East Bridgewater, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: DEPUY SPINE, INC., Raynham, MA (US)

(21) Appl. No.: 11/459,173

(22) Filed: Jul. 21, 2006

(51) Int. Cl. A61F 2/30

(52) U.S. Cl. 606/61

(57) ABSTRACT

Methods and devices are provided for connecting a spinal fixation construct to the spine, and preferably to the ilium and/or sacrum. In one exemplary embodiment, a spinal connector is provided having an elongate configuration with opposed thru-bores formed therein. Each thru-bore can be configured to receive a bone screw for attaching the spinal connector to bone. The spinal connector can also include a receiving portion formed thereon or removably mated thereto for mating a spinal fixation element, such as a spinal rod, to the spinal connector. In certain exemplary embodiments, the receiving portion can be positioned between the opposed thru-bores. In use, the spinal connector can be implanted in the sacrum and/or ilium and it can receive a laterally or horizontally extending spinal fixation element therethrough. The lateral spinal fixation element can mate to a longitudinal spinal fixation element which is mated to one or more vertebrae in a patient's spine, thereby anchoring a construct to the sacrum and/or ilium.

### FIELD OF THE INVENTION

The present invention relates to spinal connectors for mating a spinal fixation element to bone.

### BACKGROUND OF THE INVENTION

Spinal deformities, which include rotation, angulation, and/or curvature of the spine, can result from various disorders, including, for example, scoliosis (abnormal curvature in the coronal plane of the spine), kyphosis (backward curvature of the spine), and spondylolisthesis (forward displacement of a lumbar vertebra). Other causes of an abnormally shaped spine include trauma and spinal degeneration with advancing age. Early techniques for correcting such deformities utilized external devices that applied force to the spine in an attempt to reposition the vertebrae. These devices, however, resulted in severe restriction and in some cases immobility of the patient. Furthermore, current external braces have limited ability to correct the deformed spine and typically only prevent progression of the deformity. Thus, to avoid this need, doctors developed several internal fixation techniques to span across multiple vertebrae and force the spine into a desired orientation.

To fix the spine, surgeons attach one or more fixation elements (typically rods or plates) to the spine at several fixation sites, typically in the lumbar and sacral region, to correct and stabilize the spinal deformity, prevent reoccurrence of the spinal deformity, and stabilize weakness in trunks that results from degenerative discs and joint disease, deficient posterior elements, spinal fracture, and other debilitating problems. Where rods are used, they may be pre-curved or curved intraoperatively to a desired adjusted spinal curvature. Wires as well as bone screws or hooks can be used to pull individual vertebra or bone structure toward the rod, thereby anchoring the device to bone. The procedure may also include fusion of the instrumented spinal segments.

Once anchored, the rod-based systems are under stress and subjected to significant forces, known as cantilever pullout forces. As a result, surgeons are always concerned about the possibility of the implant loosening or the bone screws pulling out of the bone, especially where the system is anchored to the sacrum or ilium. The sacrum and ilium are usually of poor bone quality, consisting primarily of cancellous bone with thin cortical bone, magnifying the problem when fixation elements must be fixed to them. Thus, surgeons generally seek to attach implants in the most secure and stable fashion possible while at the same time addressing a patient's specific anatomy. While several current techniques exists for anchoring fixation elements to the sacrum and ilium, the current techniques require precise contouring and placement of spinal rods on the sacrum and/or ilium during surgery. The task becomes more difficult when, as is often called for, a surgeon must construct a framework of articulated spinal rods. As a result, while several different rod-based systems have been developed, they can be cumbersome, requiring complicated surgical procedures with long operating times to achieve correction. Furthermore, intraoperative adjustment of rod-based systems can be difficult and may result in loss of mechanical properties due to multiple bending operations. Surgeons find a number of the current techniques to be complex and challenging to implement.

Accordingly, there is a need in this art for novel implantable devices for correcting spinal deformities or degeneration that reduce the complexity of surgery, are compatible with current surgical techniques, can be easily and intraoperatively customized, and have high resistance to implant pullout.

### SUMMARY OF THE INVENTION

The present invention generally provides various implantable devices and methods for correcting spinal deformities or degeneration. In one embodiment, a spinal connector is provided and includes a spinal fixation plate having first and second thru-bores formed therethrough and configured to receive bone screws for mating the spinal fixation plate to bone. In an exemplary embodiment, the first and second thru-bores are formed on opposed ends of the spinal fixation plate. The spinal connector can also include a rod-receiving head formed on the fixation plate and positioned between the first and second thru-bores. The rod-receiving head can include opposed sidewalls defining a rod-receiving portion therebetween configured to seat a spinal fixation rod.

While the rod-receiving head can have a variety of configurations, in one exemplary embodiment the rod-receiving head can have a closed configuration. For example, the rod-receiving portion can include an opening extending through the rod-receiving head. The opening can have an axis that extends substantially parallel to a plane of the spinal fixation plate. The opening can also optionally include a collet adapted to be disposed therein and configured to receive a spinal rod therethrough. The device can further include a locking mechanism that is adapted to lock the collet with a spinal rod extending therethrough within the opening, thereby mating a spinal rod to the spinal fixation plate.

In another embodiment, the rod-receiving head can have an open configuration. For example, the head can be substantially U-shaped with opposed arms that define the rod-receiving portion therebetween. The device can also include a locking mechanism that is adapted to engage the opposed arms to lock a spinal rod within the rod-receiving portion, thereby mating a spinal rod to the spinal fixation plate.

In yet another embodiment, a spinal connector is provided having a spinal fixation plate with first and second thru-bores formed therethrough and configured to receive bone screws for mating the spinal fixation plate to bone, and a protrusion positioned between the first and second thru-bores. A head configured to polyaxially mate to the protrusion is formed on the spinal fixation plate, and it can have a rod-receiving portion configured to receive a spinal rod to mate the spinal rod to the spinal fixation plate. The head can include, for example, an opening formed in a bottom portion thereof and configured to receive the protrusion. The device can also include a locking mechanism configured to engage the protrusion to mate the head to the spinal fixation plate. For example, the protrusion can include a groove formed around a perimeter thereof, and the locking mechanism can be configured to engage the groove. The head can also include an opening formed therein for receiving the protrusion. The opening can define the rod-receiving portion. In another embodiment, the protrusion can be removably mated to the spinal fixation plate.

In other aspects, a spinal connector is provided having a spinal fixation plate with first and second thru-bores formed therethrough and configured to receive bone screws for mating the spinal fixation plate to bone, and a head positioned between the first and second thru-bores and having a rod-receiving opening extending therethrough and configured to receive a spinal rod such that the spinal rod extends at an angle transverse to a longitudinal axis of the spinal plate, and transverse to an axis that is perpendicular to the longitudinal axis of the spinal plate. In one embodiment, the head can be fixedly formed on the spinal fixation plate, and the first and second thru-bores can be formed on opposed ends of the spinal fixation plate.

A spinal fixation system is also provided and in one exemplary embodiment the system can include a spinal plate having at least one thru-bore formed therethrough and adapted to receive a bone screw for anchoring the spinal plate to bone, a first elongate spinal fixation element having a terminal portion mated to a rod-receiving member on the spinal plate, a second elongate spinal fixation element extending transverse to the first elongate spinal fixation element, and a connector mating the first and second spinal fixation elements. The spinal plate can have various configurations, but in one embodiment it can include first and second thru-bores formed therethrough with the rod-receiving member being positioned between the first and second thru-bores. The rod-receiving member can be fixedly formed on or removably mated to the spinal fixation plate. In other embodiments, the second elongate member can be configured to extend longitudinally between a plurality of vertebrae along a portion of a length of a spinal column, and the system can include several anchors for anchoring the second elongate member to a plurality of vertebrae. In yet another embodiment, the connector can be a rod-receiving member formed on a second terminal portion of the first elongate spinal fixation element. The rod-receiving member can be configured to seat the second elongate spinal fixation element to mate the second elongate spinal fixation element to the first elongate spinal fixation element. The system can also optionally include a second spinal plate having at least one thru-bore formed therethrough and adapted to receive a bone screw for anchoring the spinal plate to bone. A second terminal portion of the first elongate spinal fixation element can be mated to a rod-receiving member on the second spinal plate.

Exemplary methods for correcting spinal deformities are also provided, and in one embodiment the method can include anchoring a spinal plate to iliac or sacral bone using at least one bone screw positioned through at least one thru-bore formed in the spinal plate and threaded into bone, and mating a first end of a first spinal fixation element to a receiving member on the spinal plate. Anchoring the spinal plate can include, for example, inserting first and second bone screws through first and second thru-bores formed in the spinal plate and threading the first and second bone screws into iliac or sacral bone. In an exemplary embodiment, the first end of the first spinal fixation element is mated to the spinal plate at a location between the first and second thru-bores. The method can also include anchoring a second spinal fixation element to a plurality of vertebrae such that the second spinal fixation element extends between the plurality of vertebrae along a portion of a length of a spinal column, and extends transverse to the first spinal fixation element. The first and second spinal fixation elements can also be mated to one another.

### BRIEF DESCRIPTION OF THE DRAWINGS

The invention will be more fully understood from the following detailed description taken in conjunction with the accompanying drawings, in which:

FIG. 1A is a perspective view of one embodiment of a spinal connector having opposed thru-bores for receiving bone screws, and a rod-receiving portion for seating a spinal fixation rod extending substantially perpendicular to a longitudinal axis of the spinal connector;

FIG. 1B is a top view of another embodiment of a spinal connector having opposed thru-bores for receiving bone screws, and a rod-receiving portion for seating a spinal fixation rod extending substantially parallel to a longitudinal axis of the spinal connector;

FIG. 1C is a perspective view of yet another embodiment of a spinal connector having opposed thru-bores for receiving bone screws, and a rod-receiving portion for seating a spinal fixation rod extending at an acute angle relative to a longitudinal axis of the spinal connector

FIG. 2A is an exploded top perspective view of another embodiment of a spinal connector having opposed thru-bores for receiving bone screws, and a removable rod-receiving portion for seating a spinal fixation rod;

FIG. 2B is an exploded bottom perspective view of the spinal connector of FIG. 2A;

FIG. 2C is an exploded perspective view of the spinal connector of FIG. 2A showing first and second bone screws, first and second locking mechanisms, and a spinal fixation element configured to mate to the spinal connector;

FIG. 2D is an assembled perspective view of the system shown in FIG. 2C;

FIG. 3 is an exploded perspective view of yet another embodiment of a spinal connector having opposed thru-bores for receiving bone screws, and a rod-receiving portion having a closed configuration with an opening extending therethrough for receiving a spinal fixation rod;

FIG. 4 illustrates one exemplary embodiment of a spinal construct having longitudinal spinal fixation rods that are anchored to the sacrum using first and second spinal connectors;

FIG. 5A illustrates one exemplary embodiment of a spinal construct having longitudinal spinal fixation rods that are anchored to the ilium using first and second spinal connectors;

FIG. 5B illustrates another exemplary embodiment of a spinal construct having longitudinal spinal fixation rods that are anchored to the ilium using first and second spinal connectors; and

FIG. 6 illustrates one exemplary embodiment of a spinal construct having longitudinal spinal fixation rods that are anchored to the sacrum and ilium using four spinal connectors.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention generally provides spinal connectors for connecting a spinal fixation construct to the spine, and preferably to the ilium and/or sacrum. The spinal connectors provide a secure sacral and/or iliac connection that can be configured to counteract the cantilever pullout forces that are the cause of implant loosening in spinal constructs. For example, in one embodiment a spinal connector is provided having an elongate configuration with opposed thru-bores formed therein. Each thru-bore can be configured to receive a bone screw for attaching the spinal connector to bone. The spinal connector can also include a receiving portion formed thereon or removably mated thereto for mating a spinal fixation element, such as a spinal rod, to the spinal connector. In certain exemplary embodiments, the receiving portion can be positioned between the opposed thru-bores. In use, the spinal connector can be implanted in the sacrum and/or ilium and it can receive a spinal fixation element therein. A laterally extending spinal fixation element, for example, can mate to a longitudinal spinal fixation element which is mated to one or more vertebrae in a patient's spine, thereby anchoring a spinal construct to the sacrum and/or ilium. A person having ordinary skill in the art will appreciate that while the spinal connectors are particularly useful for anchoring a spinal construct to the sacrum or ilium, the spinal connectors and methods disclosed herein can be used in various portions of the spinal column and are not limited to use in the cervical spine.

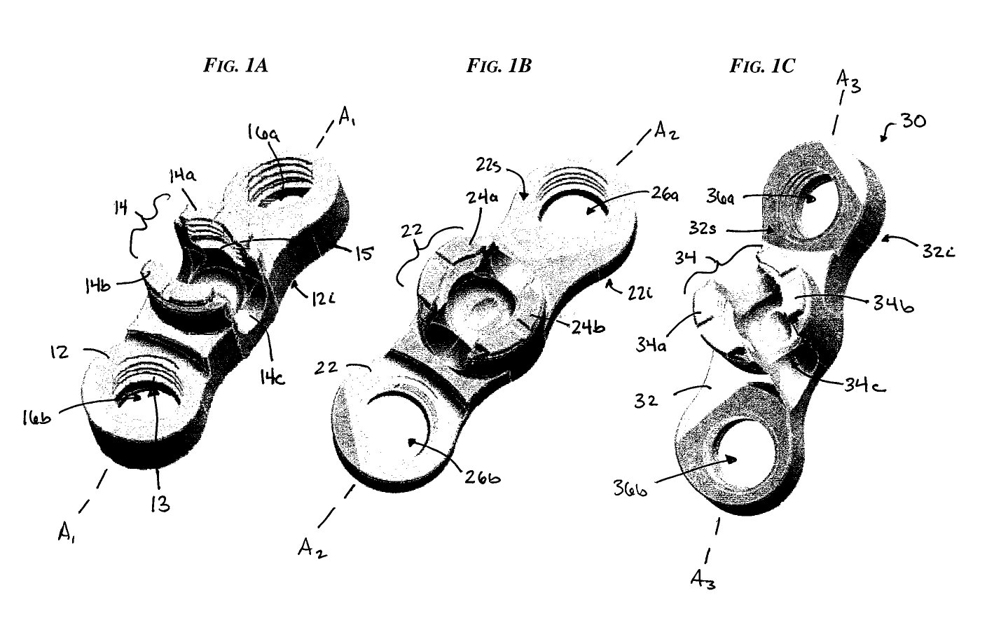

FIGS. 1A-1C illustrate various exemplary embodiments of a spinal connector 10, 20, 30 for mating a spinal fixation element, such as a spinal rod, to bone. As shown, each spinal connector 10, 20, 30 generally includes a plate 12, 22, 32 having a generally elongate configuration with opposed superior and inferior surfaces 12s, 12i, 22s, 22i, 32s, 32i. The superior surface 12s, 22s, 32s of each plate 12, 22, 32 can be configured to mate a spinal fixation element to the plate 12, 22, 32, as will be discussed below, and the inferior surface 12i, 22i, 32i of each plate 12, 22, 32 can be configured to be positioned adjacent to bone. The thickness of the plate 12, 22, 32, as measured between the superior and inferior surfaces 12s, 12i, 22s, 22i, 32s, 32i, can vary depending on the intended use, but in an exemplary embodiment each plate 12, 22, 32 preferably has a thickness that is sufficient to provide structural stability and rigidity to the plate 12, 22, 32. The length and width of each plate 12, 22, 32 can also vary depending on the intended use. For example, in an exemplary embodiment the length and width of each plate 12, 22, 32 can be adapted to allow each plate 12, 22, 32 to be implanted in iliac or sacral bone. The shape of each plate 12, 22, 32 can also vary. For example, the inferior surface 12i, 22i, 32i of each plate 12, 22, 32 can have a shape that contours the shape of a bone surface on which the plate 12, 22, 32 is configured to be positioned. A person skilled in the art will appreciate that the spinal connectors 10, 20, 30 can have a variety of configurations. Moreover, the spinal connectors 10, 20, 30 can be provided as part of a kit containing connectors of varying shapes and sizes to allow a surgeon to select the shape and size needed based on the anatomy of the patient.

As further shown in FIGS. 1A-1C, each spinal connector 10, 20, 30 can also include one or more thru-bores formed in the plate 12, 22, 32 for receiving a bone-engaging element to anchor the spinal connector 10, 20, 30 to bone. In the illustrated embodiments, each spinal connector 10, 20, 30 includes first and second thru-bores 16a, 16b, 26a, 26b, 36a, 36b formed in opposed ends of the plate 12, 22, 32. In use, a bone screw or other bone-engaging element can be inserted through each thru-bore 16a, 16b, 26a, 26b, 36a, 36b to anchor the spinal connector 10, 20, 30 to bone, thereby anchoring a spinal fixation element attached to the receiving portion 14, 24, 34 to bone. A person skilled in the art will appreciate that each plate 12, 22, 32 can have virtually any shape with any number of thru-bores or other features formed therein or thereon for anchoring the spinal connector to bone. Moreover, other techniques can be used to mate the plate 12, 22, 32 to bone. For example, each plate 12, 22, 32 can have a bone-engaging element, such as a screw, staple, etc., integrally formed thereon.

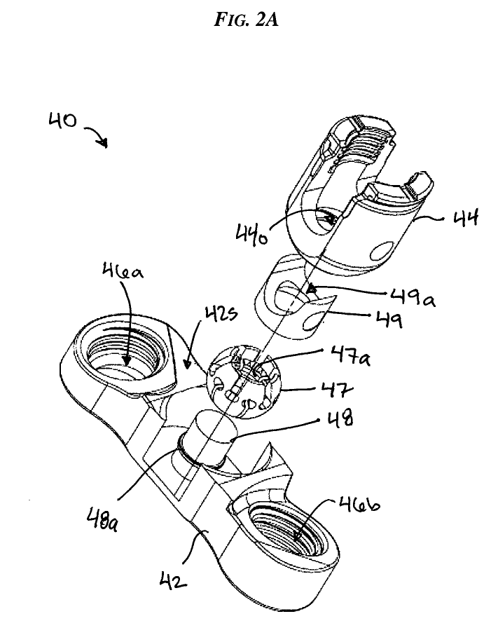

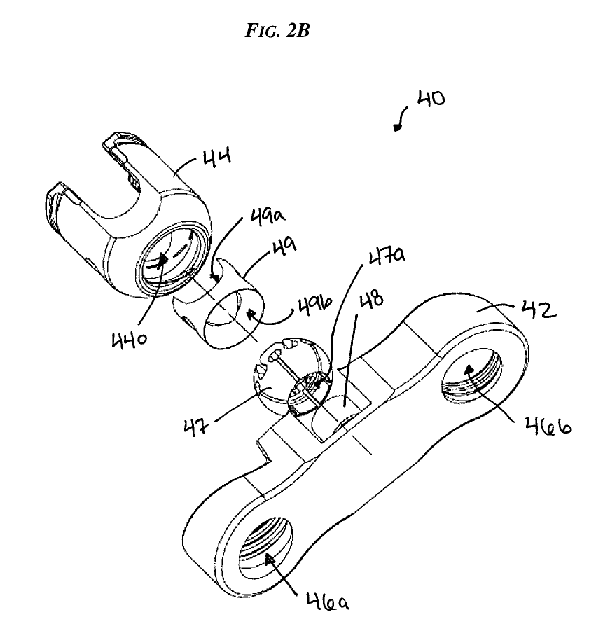

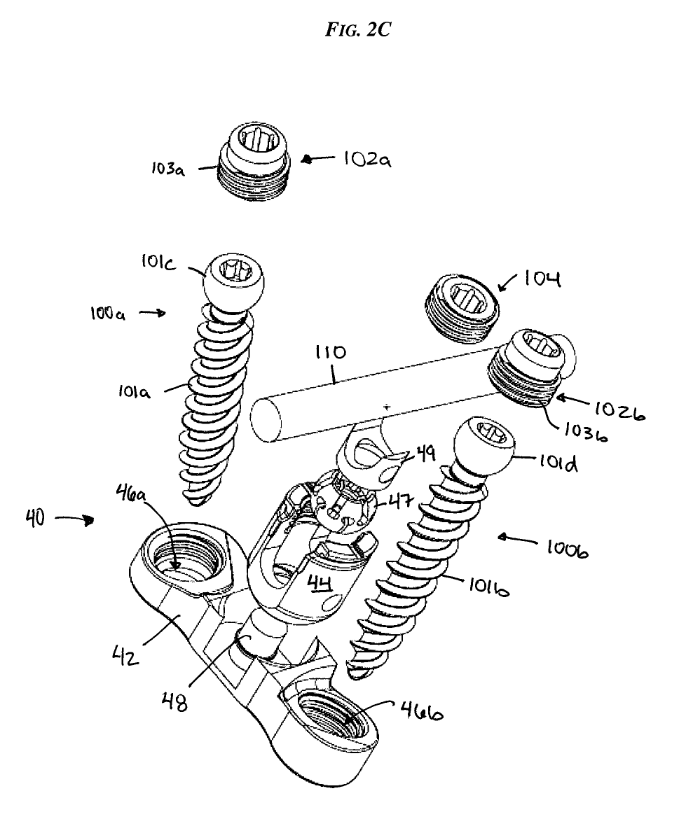

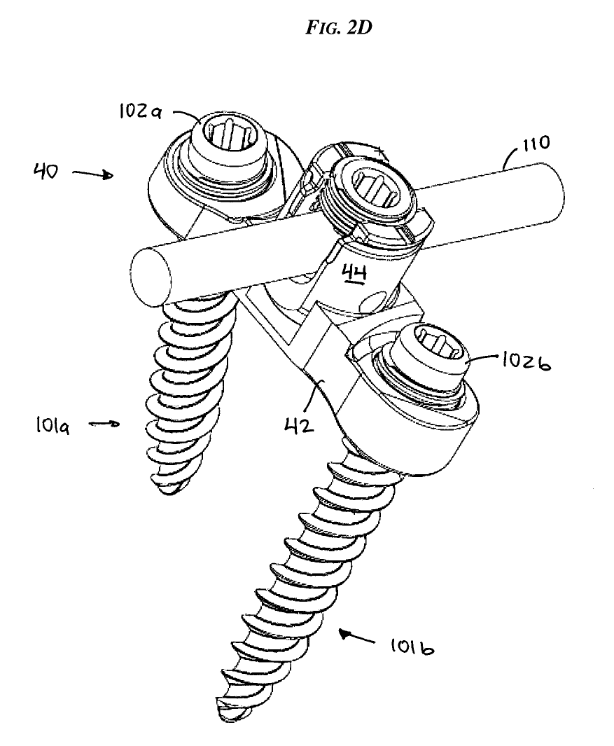

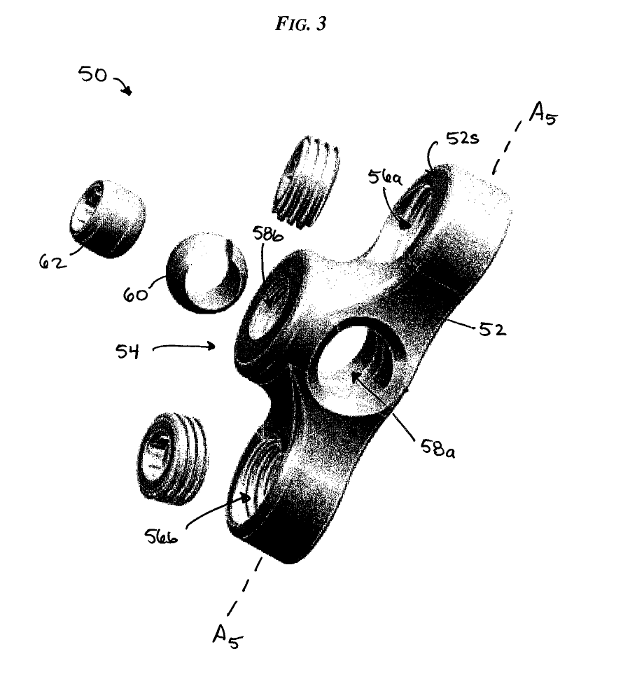

While the particular configuration of each thru-bore 16a, 16b, 26a, 26b, 36a, 36b can vary, in an exemplary embodiment the thru-bores 16a, 16b, 26a, 26b, 36a, 36b are designed to allow the bone screws to be inserted therethrough at an angle relative to a central axis of the thru-bore 16a, 16b, 26a, 26b, 36a, 36b. This is particularly advantageous as it can allow two bone screws inserted through the two thru-bores to be oriented toward one another to prevent pullout once implanted. In order to allow angular orientation of the bone screws, the thru-bores 16a, 16b, 26a, 26b, 36a, 36b can optionally include a concave surface formed around at least an interior portion thereof for seating a convex head formed on a bone screw. FIG. 1A, for example, illustrates a concave surface 13 formed within a region of thru-bore 16b that is located adjacent to the inferior surface 12i of the plate 12. The concave surface 13 is configured to pivotally or polyaxially seat the head of a bone screw. Once the bone screw is implanted in bone, a locking mechanism, such as a threaded nut, can be disposed within and threadably mated to the thru-bore 16b to lock the bone screw relative to the spinal connector 10. As shown in FIG. 1A, a region of the thru-bore 16b that is located adjacent to the superior surface 12s of the plate 12 is threaded for mating with a locking mechanism. Exemplary locking mechanisms will be discussed in more detail below with respect to FIGS. 2C and 2D. A person skilled in the art will appreciate that other techniques can be used to lock the bone screw or other bone-engaging element to the spinal connector.

FIGS. 1A-1C also illustrate a receiving portion 14, 24, 34 formed on the superior surface 12s, 22s, 32s of each plate 12, 22, 32 for mating a spinal fixation element to the spinal connector 10, 20, 30. Each receiving portion 14, 24, 34 is shown positioned between the first and second thru-bores 16a, 16b, 26a, 26b, 36a, 36b, however the position can vary depending on the configuration of each plate 12, 22, 32. For example, in other embodiments the plates 12, 22, 32 can have a triangular or square configuration and the receiving portions 14, 24, 34 can be positioned in the middle of the plate. The particular configuration of the receiving portion 14, 24, 34 on each spinal connector 10, 20, 30 can also vary depending on the type of spinal fixation element being mated thereto. In the illustrated embodiments, each receiving portion 14, 24, 34 is configured to seat a spinal rod. In particular, each receiving portion 14, 24, 34 has a generally U-shaped configuration with opposed arms 14a, 14b, 24a, 24b, 34a, 34b that extend outward from the superior surface 12s, 24s, 34s of the plate 12, 22, 32, and that define a U-shaped recess 14c, 24c, 34c therebetween. A spinal rod can be positioned between the opposed arms 14a, 14b, 24a, 24b, 34a, 34b and within the U-shaped recess 14c, 24c, 34c. The direction in which the spinal rod extends relative to a longitudinal axis A₁, A₂, A₃ that extends along a length between the opposed thru-bores 16a, 16b, 26a, 26b, 36a, 36b of the plate 12, 22, 32 of each spinal connector 10, 20, 30 can vary. In the embodiment shown in FIG. 1A, the arms 14a, 14b are substantially parallel to one another and they extend substantially perpendicular to the longitudinal axis A₁ of the plate 12, such that a spinal rod disposed within the recess 14c will extend in a direction that is substantially perpendicular to the longitudinal axis A₁ of the plate 12. In the embodiment shown in FIG. 1B, the arms 24a, 24b are substantially parallel to one another and they extend substantially parallel to the longitudinal axis A₂ of the plate 22, such that a spinal rod disposed within the recess 24c will extend parallel to the longitudinal axis A₂ of the plate 22. In the embodiment shown in FIG. 1C, the arms 34a, 34b extend substantially parallel to one another and they extend at an angle of about 45° relative to the longitudinal axis A₃ of the plate 32, such that a spinal rod disposed within the recess 34c will extend at an angle of about 45° relative to the longitudinal axis A₂ of the plate 22. A person skilled in the art will appreciate that the particular orientation can vary as desired. In other embodiments, the receiver portion 14, 24, 34 can be configured to rotate to allow a user to select a desired orientation.

Once a spinal rod or other spinal fixation element is positioned within the receiver portion 14, 24, 34, a locking mechanism can be applied to the receiver portion 14, 24, 34 to lock the spinal fixation element therein. While various locking techniques can be used, in the embodiments shown in FIGS. 1A-1C each receiver portion 14, 24, 34 includes threads formed on an interior surface of a proximal portion of each arm 14a, 14b, 24a, 24b, 34a, 34b for mating with corresponding threads on a locking mechanism. For example, FIG. 1A illustrates threads 15 formed on the interior surface of arm 14a adjacent to the terminal end thereof. The locking mechanism can be, for example, a threaded nut or set screw that can threadably mate to the arms 14a, 14b, 24a, 24b, 34a, 34b to apply a downward pressure on the spinal rod thereby locking the spinal rod within the recess 14c, 24c, 34c of the receiver portion 14, 24, 34. As a result, the spinal rod is prevented from sliding and rotating relative to the spinal connector 10, 20, 30, and is thereby anchored to the bone to which the spinal connector 10, 20, 30 is attached. Exemplary locking mechanisms will be discussed in more detail below with respect to FIGS. 2C and 2D. A person skilled in the art will appreciate that various locking mechanisms, such as snap-lock and twist-lock mechanisms, are known in the art and can be used with the spinal connectors disclosed herein. Moreover, the locking mechanism can mate to an external surface of each arm, or to both external and internal portions of each arm.

In other embodiments, the receiver portion can be removably mated to the plate of the connector. By way of non-limiting example, FIGS. 2A and 2B illustrate one such embodiment of a spinal connector 40 for anchoring a spinal fixation element to bone. In general, the spinal connector 40 includes a spinal fixation plate 42 having first and second thru-bores 46a, 46b formed therethrough, similar to the plates 12, 22, 32 described above in connection with spinal connectors 10, 20, 30. As further shown, the plate 42 includes a protrusion 48 formed thereon between the first and second thru-bores 46a, 46b. The protrusion 48 is configured to receive a receiver head 44 for mating a spinal fixation element to the plate 42. As shown in FIGS. 2A and 2B, the receiver head 44 is similar to the receiver portions 14, 24, 34 shown in FIGS. 1A-1C, however in this embodiment the receiver head 44 includes an opening 44o formed in a bottom or inferior surface thereof. The opening 44o allows the receiver head 44 to be disposed around the protrusion 48 and positioned on the superior surface 42s of the plate 42.

In order to mate the receiver head 44 to the protrusion 48, the connector 40 can include an expandable collet 47 that is disposable around the protrusion 48, and that expands and contracts to engage the protrusion 48. As shown in FIGS. 2A and 2B, the protrusion 48 includes a ridge 48a formed there around and the collet 47 includes a groove 47a formed therein and configured to seat the ridge 48a on the protrusion 48. The connector 40 can also include an insert 49 that is disposable within the receiver head 44, and that includes an inferior portion with a concave cavity 49b formed therein for seating a proximal portion of the collet 47, and a superior portion with a U-shaped recess 49a formed therein for seating a spinal fixation element, such as a spinal rod. In use, the collet 47 is disposed around and loosely mated to the protrusion 48, the receiver head 44 is positioned around the collet 47, and the insert 49 is positioned within the receiver head 44 such that the collet 47 is seated within the concave cavity 49b formed in the inferior portion of the insert 49. Prior to locking a spinal fixation element within the receiver head 44, the receiver head 44, insert 49, and optionally the collet 47 are free to rotate relative to the protrusion 48. This will allow the U-shaped recess 49a to be positioned at any orientation relative to a longitudinal axis A₄ of the plate 42, thereby allowing a spinal fixation element disposed within the receiver head and seated in the recess 49a to be positioned at various angles relative to the longitudinal axis A₄ of the plate 42.

Once a spinal fixation element is disposed within the receiver head 44 and seated in the U-shaped recess 49a of the insert 49, a locking mechanism can be applied to the receiver head 44 to lock the spinal fixation element therein. When this occurs, the force applied to the spinal fixation element by the locking mechanism will be applied to the insert 49, thereby moving the insert downward toward the protrusion 48. As a result, the insert 49 will apply pressure to the collet 47 causing the collet 47 to contract or decrease in diameter and fixedly engage the protrusion 48. The collet 47 will also apply pressure to the portion of the receiver head 44 that surrounds the opening 44o formed therein. The collet 47, insert 49, receiver head 44, and spinal fixation element will thus be locked in a fixed position relative to the plate 42 of the connector 40.

By way of non-limiting example, FIGS. 2C and 2D illustrate bone-engaging elements for mating the connector 40 to bone, and locking mechanisms for locking the bone-engaging elements relative to the connector 40, and for locking a spinal fixation element within the receiver head 42. Referring first to FIG. 2C, first and second bone-engaging elements in the form of bone screws 100a, 100b are shown. Each bone screw 100a, 100b includes a threaded shank 101a, 101b and a head 101c, 101d formed thereon. The shank 101a, 101b is configured to be disposed within bone, and the head 101c, 101d has a generally hemi-spherical configuration to allow the head 101c, 101d to be pivotally or polyaxially disposed within the thru-bore 46a, 46b in the plate 42 of the connector 40. FIG. 2D illustrates the bone screws 100a, 100b disposed within the thru-bores 46a, 46b. In order to lock and prevent movement of the heads 101c, 101d relative to the thru-bores 46a, 46b, and thus mate the spinal connector 40 to bone, a set screw 102a, 102b can be threadably mated to each thru-bore 46a, 46b. As shown in FIG. 2C, each set screw 102a, 102b includes a threaded distal portion 103a, 103b that is configured to engage and mate to the threads formed within the thru-bore 46a, 46b.

FIGS. 2C and 2D also illustrate a spinal fixation rod 110 that mates to the receiver head 44. When the rod 110 is positioned within the receiver head 44, a locking mechanism can be applied to the receiver head 44 to lock the rod 110 therein. In the illustrated embodiment, the locking mechanism is a set screw 104 having a configuration similar to the set screws 102a, 120b previously described above. The set screw 104 includes threads formed thereon for mating with threads formed within the receiver head 44. Once mated, the set screw 104 will lock the spinal rod 110 and the receiver head 44 in a fixed position relative to the plate 42, as previously explained.

In other embodiments, rather than having a receiver portion or head with an open configuration that has opposed arms that define a U-shaped recess for receiving a spinal fixation element, the receiver portion or head can have a closed configuration with a bore or opening extending therethrough for receiving a spinal fixation element. By way of non-limiting example, FIG. 3 illustrates one such exemplary embodiment of a spinal connector 50 having a receiver portion 54 with a closed configuration. As shown, the spinal connector 50 is similar to the connectors 10, 20, 30 shown in FIGS. 1A-1C and includes a plate 52 having first and second thru-bores 56a, 56b formed therein. A receiver portion 54 is formed on the plate 52 between the thru-bores 56a, 56b, and it has a generally cylindrical configuration that extends outward from the superior surface 52s of the plate 52. A first bore or opening 58a is formed through a mid-portion of the plate 52 and through opposed sidewalls of the receiver portion 54, and it extends in a direction that is substantially perpendicular to a longitudinal axis A₅ that extends between the thru-bores 56a, 56b of the plate 52. While the shape and size of the first opening 58a can vary, in the illustrated embodiment the opening 58a is substantially cylindrical and is sized to slidably receive a cylindrical spinal rod therethrough. The receiver portion 54 can also be configured to receive a locking mechanism for locking the spinal fixation element relative to the spinal connector 50. In the illustrated embodiment, the receiver portion 54 includes a second opening 58b formed in a superior surface thereof and extending into the first opening 58a such that the first and second openings 58a, 58b intersect. A screw, nut, or other locking mechanism, such as the set screw 62 shown in FIG. 3, can be disposed within and mated to the second opening 58b to lock a spinal fixation element in the first opening 58a. While not shown, the set screw 62 can include threads or other features formed thereon for mating with corresponding features formed within the receiver portion 54.

While pressure from the locking mechanism can be sufficient to lock the spinal fixation element within the first opening 58a, in an exemplary embodiment the spinal connector 50 can also include a clamp, such as a split o-ring 60, that sits within the first opening 58a and receives the spinal fixation element therethrough. As shown in FIG. 3, the split o-ring 60 can be oriented such that its inner lumen is aligned with the inner lumen of the first opening 58a. When the locking mechanism is applied to the second opening 58b, the locking mechanism will apply pressure to the split o-ring 60, thereby causing the split o-ring 60 to close and engage the spinal fixation element to lock it within the first opening 58a. A person having ordinary skill in the art will appreciate that a variety of other techniques can be used to lock a spinal fixation element to the spinal connector.

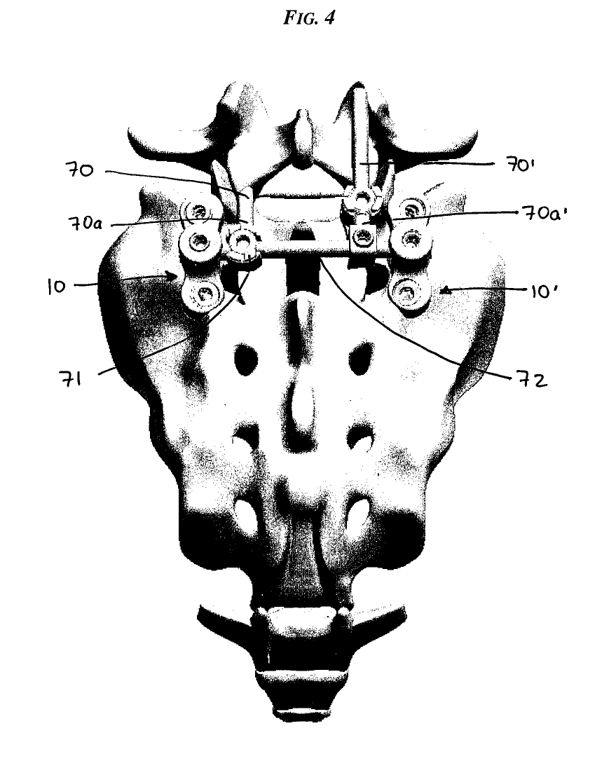

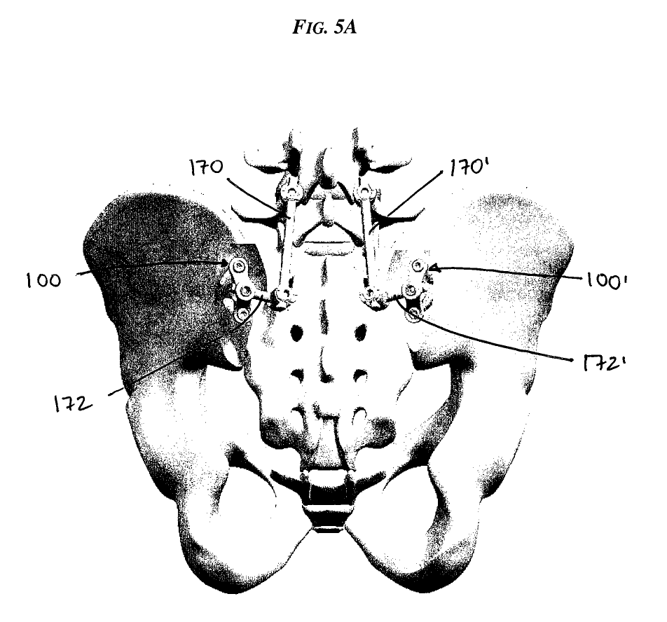

FIGS. 4-7 illustrates various exemplary spinal fixation constructs that utilize a spinal connector for anchoring the construct to the sacrum and/or ilium. While the methods and constructs are described in connection with the spinal connector 10 shown in FIG. 1A, a person skilled in the art will appreciate that the spinal connector can have virtually any configuration, and that the particular configuration can vary depending on the intended use. Moreover, the components used in each construct and the particular configuration of each component can vary. Various other devices known in the art can also be used to provide certain mating connections between the components of the various constructs.

Turning first to FIG. 4, one exemplary construct for sacral fixation is shown and includes first and second spinal connectors 10, 10′ that are implanted in the sacrum and that are used to mate a lateral spinal fixation element to first and second longitudinal spinal fixation elements. In order to implant each spinal connector 10, 10′ in the sacrum, the sacral bone is prepared using standard procedures known in the art and first and second bone-screws are disposed through the thru-bores in the plate of each connector 10, 10′. Pre-drilled bone holes can be used to facilitate implantation of the bone screws. In an exemplary embodiment, the bone screws are angled toward one another to prevent back-out and to thus provide a secure mating connection between the spinal connectors 10, 10′ and the sacrum. As further shown in FIG. 4, first and second longitudinal spinal fixation elements, such as spinal rods 70, 70′ can be positioned to extend along at least a portion of a length of the spinal column such that the rods 70, 70′ span across several vertebrae. Each rod 70, 70′ can be anchored to the vertebrae using various anchoring techniques known in the art. As further shown in FIG. 4, a terminal end 70a, 70a′ of each rod 70, 70′ can be mated to a lateral member for connecting the rods 70, 70′ to the spinal connectors 10, 10′ and thereby anchoring the construct to the sacrum. While the lateral member can have various configurations, in the illustrated embodiment the lateral member is in the form of a spinal rod 72 that extends between the first and second spinal connectors 10, 10′. The terminal ends of the lateral spinal rod 72 can mate to the connectors 10, 10′ by positioning the ends within the receiver portion of each connector 10, 10′ and locking the rod 72 therein using a locking mechanism as previously described. The construct can also include another connector for mating the lateral rod 72 to the longitudinal rods 70, 70′, or the longitudinal rods 70, 70′ can include a mating element formed on a terminal end thereof for mating directly to the lateral spinal rod 72. As shown in FIG. 4, the first longitudinal rod 70 is in the form of a roded-connector. In particular, the terminal end 70a includes a receiver head 71 formed thereon and configured to receive the lateral spinal rod 72 therein. The connector is described in more detail in a U.S. patent application filed on even date herewith and entitled “Articulating Sacral or Iliac Connector,” by Nam T. Chao et al. (Attorney Docket No. 101896-470), which is hereby incorporated by reference in its entirety. The second longitudinal rod 70′, on the other hand, is mated to the lateral spinal rod 72 using a connector.

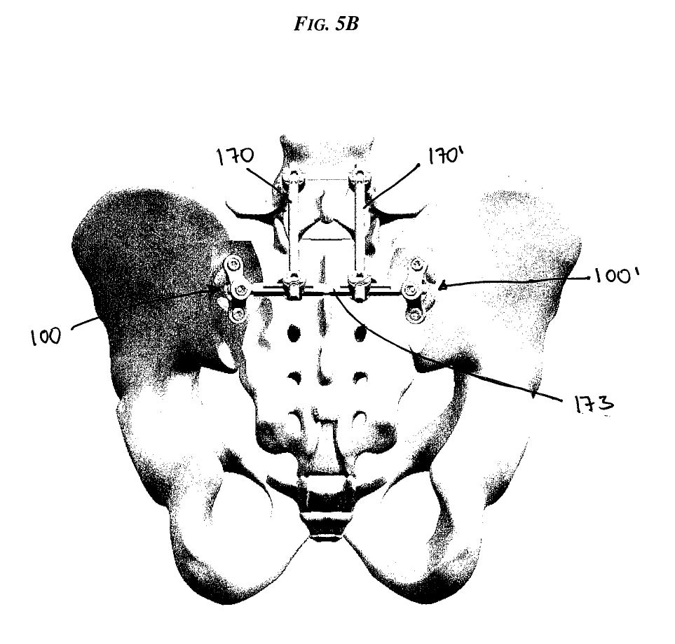

FIGS. 5A and 5B illustrate one exemplary construct for iliac fixation. In the embodiment shown in FIG. 5A, the first and second spinal connectors 100, 100′ are implanted in the ilium, and they are mated to first and second longitudinal spinal fixation elements 170, 170′ using first and second lateral connectors 172, 172′, which are described in more detail in a U.S. patent application filed on even date herewith and entitled “Articulating Sacral or Iliac Connector,” by Nam T. Chao et al. (Attorney Docket No. 101896-470), which is hereby incorporated by reference in its entirety. In general, each lateral connector 172, 172′ includes a receiver head that mates to a terminal end of the longitudinal rod 170, 170′, and a spinal rod extending from the receiver head and that mates to the receiver portion on the spinal connector 100, 100′ implanted in the ilium. In the embodiment shown in FIG. 5B, the first and second lateral connectors 172, 172′ of FIG. 5A are replaced with a single cross-connector 173, which is described in more detail in a U.S. patent application filed on even date herewith and entitled “Sacral or Iliac Cross Connector,” by Nam T. Chao et al. (Attorney Docket No. 101896-471), which is hereby incorporated by reference in its entirety. In general, the cross-connector 173 includes first and second receiver heads slidably disposed along a spinal fixation element or rod. Each receiver head is effective to mate to a terminal end of the longitudinal rod 170, 170′, and to lock in a fixed position along the spinal fixation element. The terminal ends of the cross-connector 173 mate to the receiver head on the connectors 100, 100′ to thereby anchor the cross-connector 173, and thus the longitudinal rods 170, 170′ to the ilium.

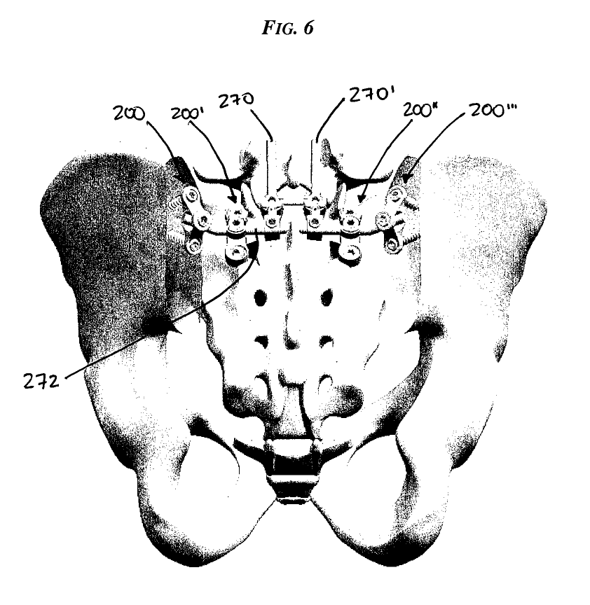

FIG. 6 illustrates yet another embodiment of a spinal construct. In this embodiment, four connectors 200, 200′, 200″, 200′″ are used to anchor first and second longitudinal spinal rods 270, 270′ to the sacrum and the ilium, i.e., for sacroiliac fixation. In particular, first and second spinal connectors 200, 200′ are implanted in the ilium, and third and fourth spinal connectors 200″, 200′″ are implanted in the sacrum. A lateral spinal rod 272 that extends laterally across the spine mates to the receiver portion on each spinal connector 200, 200′, 200″, 200′″. The lateral spinal rod 272 is also mated to a terminal end of each longitudinal rod 270, 270′ to thereby anchor the longitudinal rods 270, 270′ to the sacrum and ilium. Again, various anchoring techniques such as those previously described can be used to mate the longitudinal rods 270, 270′ to the lateral rod 272.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A spinal connector, comprising:
a spinal fixation plate having first and second thru-bores formed therethrough and configured to receive bone screws for mating the spinal fixation plate to bone; and
a rod-receiving head formed on the fixation plate and positioned between the first and second thru-bores, the rod-receiving head including opposed sidewalls defining a rod-receiving portion therebetween configured to seat a spinal fixation rod.

2. The spinal connector of claim 1, wherein the first and second thru-bores are formed on opposed ends of the spinal fixation plate.

3. The spinal connector of claim 1, wherein the rod-receiving portion comprises an opening extending through the rod-receiving head.

4. The spinal connector of claim 3, wherein the opening has an axis that extends substantially parallel to a plane of the spinal fixation plate.

5. The spinal connector of claim 3, further comprising a collet adapted to be disposed within the opening and configured to receive a spinal rod therethrough.

6. The spinal connector of claim 5, further comprising a locking mechanism adapted to lock the collet with a spinal rod extending therethrough within the opening, thereby mating a spinal rod to the spinal fixation plate.

7. The spinal connector of claim 1, wherein the head is substantially U-shaped with opposed arms that define the rod-receiving portion therebetween.

8. The spinal connector of claim 7, further comprising a locking mechanism adapted to engage the opposed arms to lock a spinal rod within the rod-receiving portion, thereby mating a spinal rod to the spinal fixation plate.

9. A spinal connector, comprising:
a spinal fixation plate having first and second thru-bores formed therethrough and configured to receive bone screws for mating the spinal fixation plate to bone, and a protrusion positioned between the first and second thru-bores;
a head configured to polyaxially mate to the protrusion formed on the spinal fixation plate, and having a rod-receiving portion configured to receive a spinal rod to mate the spinal rod to the spinal fixation plate.

10. The spinal connector of claim 9, wherein the head includes an opening formed in a bottom portion thereof configured to receive the protrusion.

11. The spinal connector of claim 10, further comprising a locking mechanism configured to engage the protrusion to mate the head to the spinal fixation plate.

12. The spinal connector of claim 11, wherein the protrusion includes a groove formed around a perimeter thereof, and wherein the locking mechanism is configured to engage the groove.

13. The spinal connector of claim 11, wherein the head includes an opening formed therein for receiving the protrusion, the opening defining the rod-receiving portion.

14. The spinal connector of claim 9, wherein the protrusion is removably mated to the spinal fixation plate.

15. The spinal connector of claim 9, wherein the first and second thru-bores are formed on opposed ends of the spinal fixation plate.

16. A spinal connector, comprising:
a spinal fixation plate having first and second thru-bores formed therethrough and configured to receive bone screws for mating the spinal fixation plate to bone; and
a head positioned between the first and second thru-bores and having a rod-receiving opening extending therethrough and configured to receive a spinal rod such that the spinal rod extends at an angle transverse to a longitudinal axis of the spinal plate, and transverse to an axis that is perpendicular to the longitudinal axis of the spinal plate.

17. The spinal connector of claim 16, wherein the head is fixedly formed on the spinal fixation plate.

18. The spinal connector of claim 16, wherein the first and second thru-bores are formed on opposed ends of the spinal fixation plate.

19. A spinal fixation system, comprising:
a spinal plate having at least one thru-bore formed therethrough and adapted to receive a bone screw for anchoring the spinal plate to bone;
a first elongate spinal fixation element having a terminal portion mated to a rod-receiving member on the spinal plate;
a second elongate spinal fixation element extending transverse to the first elongate spinal fixation element; and
a connector mating the first and second spinal fixation elements.

20. The system of claim 19, wherein the spinal plate includes first and second thru-bores formed therethrough, and the rod-receiving member is positioned between the first and second thru-bores.

21. The system of claim 20, wherein the rod-receiving member is removably mated to the spinal fixation plate.

22. The system of claim 20, wherein the rod-receiving member is fixedly formed on the spinal fixation plate.

23. The system of claim 19, wherein the second elongate member is configured to extend longitudinally between a plurality of vertebrae along a portion of a length of a spinal column, and the system further comprises a plurality of anchors for anchoring the second elongate member to a plurality of vertebrae.

24. The system of claim 20, wherein the connector comprises a rod-receiving member formed on a second terminal portion of the first elongate spinal fixation element, the rod-receiving member being configured to seat the second elongate spinal fixation element to mate the second elongate spinal fixation element to the first elongate spinal fixation element.

25. The system of claim 20, further comprising a second spinal plate having at least one thru-bore formed therethrough and adapted to receive a bone screw for anchoring the spinal plate to bone, a second terminal portion of the first elongate spinal fixation element being mated to a rod-receiving member on the second spinal plate.

26. A method for correcting spinal deformities, comprising:
anchoring a spinal plate to iliac or sacral bone using at least one bone screw positioned through at least one thru-bore formed in the spinal plate and threaded into bone; and
mating a first end of a first spinal fixation element to a receiving member on the spinal plate.

27. The method of claim 26, wherein anchoring the spinal plate comprises inserting first and second bone screws through first and second thru-bores formed in the spinal plate and threading the first and second bone screws into iliac or sacral bone.

28. The method of claim 27, wherein the first end of the first spinal fixation element is mated to the spinal plate at a location between the first and second thru-bores.

29. The method of claim 26, further comprising anchoring a second spinal fixation element to a plurality of vertebrae such that the second spinal fixation element extends between the plurality of vertebrae along a portion of a length of a spinal column, and extends transverse to the first spinal fixation element.

30. The method of claim 29, further comprising mating the first and second spinal fixation elements to one another.
